(19) United States
(12) Patent Application Publication
(10) Pub. No.: US 2006/0210634 A1
(43) Pub. Date: Sep. 21, 2006

(54) Ceramic structures for controlled release of biologically active substances

(76) Inventors: Rudi E. Moerck, San Antonio, TX (US); Bruce J. Sabacky, Reno, NV (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/367,226

(22) Filed: Mar. 3, 2006

Related U.S. Application Data
(60) Provisional application No. 60/659,016, filed on Mar. 4, 2005.

(51) Int. Cl. A01N 25/00; A61K 9/14

(52) U.S. Cl. 424/484; 504/358; 977/906

(57) ABSTRACT

The present invention provides compositions for controlled drug delivery, dosage forms, and processes for producing dosage forms. In a composition aspect of the present invention, a composition including a drug and a ceramic structure is provided. The ceramic structure has either a hollow portion wherein the drug is included in the hollow portion or is a collection of smaller particles bound together.

### CROSS-REFERENCE TO RELATED APPLICATION

This application claims priority to U.S. Provisional Patent Application Ser. No. 60/659,016 filed on Mar. 4, 2005,the entire disclosure of which is incorporated by reference.

### FIELD OF INVENTION

The present invention generally relates to the controlled release of biologically active substances (i.e., “BAS”s). More specifically, it relates to BAS/ceramic structure combinations that provide controlled BAS release (e.g., drug delivery when administered orally).

### BACKGROUND OF INVENTION

There are many known methods directed to the administration of compositions that provide for controlled BAS release. Japanese Patent No. 2518882, for instance discusses a sustained release formulation involving pellets of inert materials that are coated with a drug-containing layer. A second coating, which includes a lipophilic compound, is laid on top of the drug-containing layer. The second layer serves as a barrier through which the drug must travel, and thereby produces a sustained release profile upon oral administration. Such compositions are difficult to produce and are easily diverted since disintegration of the second layer occurs easily during tablet compression.

Another method involves the incorporation of drugs within polymer-based, microparticle matrices. Such polymer matrices are reported in a number of patents, including U.S. Pat. No. 5,213,812, U.S. Pat. No. 5,417,986, U.S. Pat. No. 5,360,610, and U.S. Pat. No. 5,384,133. Sustained drug delivery results upon administration, since an included drug must diffuse through the matrix to reach the gastrointestinal tract of a patient. Microparticle matrices exhibit poor loading efficiencies, though, resulting in only a small percentage of incorporated drug. Additionally, the microparticle matrix delivery system is readily subverted upon crushing.

A further method is reported in U.S. Pat. No. 5,536,507. The method involves a three-component pharmaceutical formulation involving incorporation of a drug into a pH sensitive polymer that swells in regions of a patient's body exhibiting higher pHs. The formulation additionally includes a delayed release coating and an enteric coating, which affords a dosage form that releases most of the drug in the large intestine. Due to the fact that it takes several hours for the dosage form to reach the large intestine, however, widely varying time-release profiles are observed. Additionally, the three-component delivery system is readily subverted upon crushing.

There is accordingly a need for novel methods and compositions that provide for controlled BAS release (e.g., delivery). That is an object of the present invention.

### SUMMARY OF INVENTION

The present invention provides compositions for controlled release of BASs, dosage forms where the BAS is a drug, and processes for making the compositions.

In a composition aspect of the present invention, a composition including a BAS (e.g., drug, biocide, fungicide, etc.) and a ceramic structure is provided. The ceramic structure includes a metal oxide or a mixed metal oxide selected from a group consisting of titanium oxide, tantalum oxide, zirconium oxide, scandium oxide, cerium oxide and yttrium oxide.

In a dosage form aspect of the present invention where—the BAS is a drug—an oral, sustained release dosage form including a combination of a drug, a ceramic structure, and at least one polymer coating is provided.

In a process aspect of the present invention, a process for preparing a BAS/ceramic structure composition (e.g., dosage form for a drug) is provided. The process includes at least the following steps: dissolving the BAS (e.g., drug) in a solvent to provide a solution; contacting the solution with the ceramic structure; and, evaporating the solvent.

In another process aspect of the present invention, a process for preparing a BAS/ceramic structure composition (e.g., dosage form for a drug) is provided. The process includes at least the following steps: contacting a BAS (e.g., drug) melt with the ceramic structure to provide a mixture; and, allowing the mixture to cool, which affords a powder.

### DETAILED DESCRIPTION

The present invention is directed to BAS/ceramic structure combinations that provide controlled BAS release (e.g., where the BAS is a drug, drug delivery when administered orally).

The BAS may be any substance that produces a biological response within an organism (e.g., bacteria, fungus, mammal). Nonlimiting examples of BASs include drugs, biocides, fungicides, algaecides, pesticides, mildewcides, and bacteriocides.

Examples of BASs that are drugs include, without limitation, the following: antipyretics, analgesics and antiphlogistics (e.g., indoinethacin, aspirin, diclofenac sodium, ketoprofen, ibuprofen, mefenamic acid, azulene, phenacetin, isopropyl antipyrine, acetaminophen, benzadac, phenylbutazone, flufenamic acid, sodium salicylate, salicylamide, sazapyrine and etodolac); steroidal anti-inflammatory drugs (e.g., dexamethasone, hydrocortisone, prednisolone and triamcinolone); antiulcer drugs (e.g., ecabet sodium, enprostil, sulpiride, cetraxate hydrochloride, gefarnate, irsogladine maleate, cimetidine, ranitidine hydrochloride, famotidine, nizatidine and roxatidine acetate hydrochloride); coronary vasodilators (e.g., nifedipine, isosorbide dinitrate, diltiazem hydrochloride, trapidil, dipyridamole, dilazep hydrochloride, verapamil, nicardipine, nicardipine hydrochloride and verapamil hydrochloride); peripheral vasodilators (e.g., ifenprodil tartrate, cinepacide maleate, ciclandelate, cynnaridine and pentoxyfylline); antibiotics (e.g., ampicillin, amoxicillin, cefalexin, erythromycin ethyl succinate, bacampicillin hydrochloride, minocycline hydrochloride, chloramphenicol, tetracycline, erythromycin, ceftazidime, cefuroxime sodium, aspoxicillin and ritipenem acoxyl hydrate); synthetic antimicrobials (e.g., nalidixic acid, piromidic acid, pipemidic acid trihydrate, enoxacin, cinoxacin, ofloxacin, norfloxacin, ciprofloxacin hydrochloride and sulfamethoxazole-trimethoprim); antiviral agents (e.g., aciclovir and ganciclovir); anticonvulsants (e.g., propantheline bromide, atropine sulfate, oxitropium bromide, timepidium bromide, scopolamine butylbromide, trospium chloride, butropium bromide, N-methylscopolamine methylsulfate and methyloctatropine bromide); antitussives (e.g., tipepidine hibenzate, methylephedrine hydrochloride, codeine phosphate, tranilast, dextromethorphan hydrobromide, dimemorfan phosphate, clofenadol hydrochloride, fominoben hydrochloride, benproperine phosphate, eprazinone hydrochloride, clofedanol hydrochloride, ephedrine hydrochloride, noscapine, pentoxyverine citrate, oxeladin citrate and isoaminyl citrate); expectorants (e.g., bromhexine hydrochloride, carbocysteine, ethyl cysteine hydrochloride and methylcysteine hydrochloride); bronchodilators (e.g., theophylline, aminophylline, sodium cromoglicate, procaterol hydrochloride, trimetoquinol hydrochloride, diprophylline, salbutamol sulfate, clorprenaline hydrochloride, formoterol fumarate, orciprenaline sulfate, pirbuterol hydrochloride, hexoprenaline sulfate, bitolterol mesilate, clenbuterol hydrochloride, terbutaline sulfate, mabuterol hydrochloride, fenoterol hydrobromide and methoxyphenamine hydrochloride); cardiacs (e.g., dopamine hydrochloride, dobutamine hydrochloride, docarpamine, denopamine, caffeine, digoxin, digitoxin and ubidecarenone); diuretics (e.g., furosemide, acetazolamide, trichlormethiazide, methylclothiazide, hydrochlorothiazide, hydroflumethiazide, ethiazide, cyclopenthiazide, spironolactone, triamterene, florothiazide, piretanide, mefruside, etacrynic acid, azosemide and clofenamide); muscle relaxants (e.g., chlorphenesin carbamate, tolperisone hydrochloride, eperisone hydrochloride, tizanidine hydrochloride, mephenesine, chlorzoxazone, phenprobamate, methocarbamol, chlormezanone, pridinol mesilate, afloqualone, baclofen and dantrolene sodium); cerebral metabolism ameliorants (e.g., nicergoline, meclofenoxate hydrochloride and taltireline); minor tranquilizers (e.g., oxazolam, diazepam, clotiazepam, medazepam, temazepaam, fludiazepam, meprobamate, nitrazepam and chlordiazepoxide); major tranquilizers (e.g., sulpiride, clocapramine hydrochloride, zotepine, chlorpromazine and haloperidol); beta-blockers (e.g., bisoprolol fumarate, pindolol, propranolol hydrochloride, carteolol hydrochloride, metoprolol tartrate, labetanol hydrochloride, acebutolol hydrochloride, bufetolol hydrochloride, alprenolol hydrochloride, arotinolol hydrochloride, oxprenolol hydrochloride, nadolol, bucumolol hydrochloride, indenolol hydrochloride, timolol maleate, befunolol hydrochloride and bupranolol hydrochloride); antiarrthymics (e.g., procainamide hydrochloride, disopyramide phosphate, cibenzoline succinate, ajmaline, quinidine sulfate, aprindine hydrochloride, propafenone hydrochloride, mexiletine hydrochloride and ajmilide hydrochloride); athrifuges (e.g., allopurinol, probenicid, colistin, sulfinpyrazone, benzbromarone and bucolome); anticoagulants (e.g., ticlopidine hydrochloride, dicumarol, potassium warfarin, and (2R,3R)-3-acetoxy-5-[2(dimethylamino) ethyl]-2,3-dihydro-8-methyl-2-(4-ethylphenyl)-1,5-benzothiazepine-4(5H)-one maleate); thrombolytics (e.g., methyl(2E,3Z)-3-benzylidene-4-(3,5-dimethoxy-.alpha.-methyl benzylidene)-N-(4-methylpiperazin-1-yl) succinamate hydrochloride); liver disease drugs (e.g., (+)-r-5-hydroxymethyl-t-7-(3,4-dimethoxyphenyl)-4-oxo-4,5,6,7-tetrahydro benzo[b]furan-c-6-carboxylactone); antiepileptics (e.g., phenytoin, sodium valproate, metalbital and carbamazepine); antihistamines (e.g., chlorpheniramine maleate, clemastine fumarate, mequitazine, alimemazine tartrate, cyproheptadine hydrochloride and bepotastin besilate); antiemetics (e.g., difenidol hydrochloride, metoclopramide, domperidone and betahistine mesilate and trimebutine maleate); depressors (e.g., dimethylaminoethyl reserpilinate dihydrochloride, rescinnamine, methyldopa, prazocin hydrochloride, bunazosin hydrochloride, clonidine hydrochloride, budralazine, urapidil and N-[6-[2-[(5-bromo-2-pyrimidinyl)oxy]ethoxy]-5-(4-methylphenyl)-4-pyrimidinyl]-4-(2-hydroxy-1,1-dimethylethyl) benzene sulfonamide sodium);. hyperlipidemia agents (e.g., pravastatin sodium and fluvastatin sodium); sympathetic nervous stimulants (e.g., dihydroergotamine mesilate and isoproterenol hydrochloride, etilefrine hydrochloride); oral diabetes therapeutic drugs (e.g., glibenclamide, tolbutamide and glymidine sodium); oral carcinostatics (e.g., marimastat); vitamins (e.g., vitamin B1, vitamin B2, vitamin B6, vitamin B12, vitamin C and folic acid); thamuria therapeutic drugs (e.g., flavoxate hydrochloride, oxybutynin hydrochloride and terolidine hydrochloride); and, angiotensin convertase inhibitors (e.g., imidapril hydrochloride, enalapril maleate, alacepril and delapril hydrochloride).

Examples of BASs that are fungicides include: aliphatic nitrogen fungicides. (e.g., butylamine, cymoxanil, dodicin, dodine, guazatine, iminoctadine); amide fungicides (e.g., carpropamid chloraniformethan cyflufenamid, diclocymet, ethaboxam, fenoxanil, flumetover, furametpyr, mandipropamid, penthiopyrad, prochloraz, quinazamid, silthiofam, and triforine); acylamino acid fungicides (e.g., benalaxyl, benalaxyl-M, furalaxyl, metalaxyl, metalaxyl-M, and pefurazoate); anilide fungicides (e.g., benalaxyl, benalaxyl-M, boscalid, carboxin, fenhexamid, metalaxyl, metalaxyl-M, metsulfovax, ofurace, oxadixyl, oxycarboxin, pyracarbolid, thifluzamide, and tiadinil); benzanilide fungicides (e.g., benodanil, flutolanil, mebenil, mepronil, salicylanilide, tecloftalam); furanilide fungicides (e.g., fenfuram, furalaxyl, furcabanil, methfuroxam); sulfonanilide fungicides (e.g., flusulfamide); benzamide fungicides (e.g., benzohydroxamic acid, fluopicolide, tioxymid, trichlamide, zarilamid, zoxamide); furamide fungicides (e.g., cyclafuramid, furmecyclox); phenylsulfamide fungicides (e.g., dichlofluanid, tolylfluanid); antibiotic fungicides (e.g., aureofuingin, blasticidin-S, cycloheximide, griseofulvin, kasugamycin, natamycin, polyoxins, polyoxorim, streptomycin, and validamycin); strobilurin fungicides (e.g., azoxystrobin, dimoxystrobin, fluoxastrobin, kresoxim-methyl, metominostrobin, orysastrobin, picoxystrobin, pyraclostrobin, trifloxystrobin); aromatic fungicides (e.g., biphenyl, chlorodintronaphthalene, chloroneb, chlorothalonil, cresol, dicloran, hexachlorobenzene, pentachlorophenol, quintozene, sodium pentachlorophenoxide, tecnazene); benzimidazole fungicides (e.g., benomyl, carbendazim, chlorfenazole, cypendazole, debacarb, fuberidazole, mecarbinzid, rabenzazole, thiabendazole); benzimidazole precursor fungicides (e.g., fuirophanate, thiophanate, thiophanate-methyl); benzothiazole fuingicides (e.g., bentaluron, chlobenthiazone, TCMTB); bridged diphenyl fuingicides (e.g., bithionol, dichlorophen, diphenylamine); carbamate fuingicides (e.g., benthiavalicarb, furophanate, iprovalicarb, propamocarb, thiophanate, thiophanate-methyl); benzimidazolylcarbamate fuingicides (e.g., benomyl, carbendazim, cypendazole, debacarb, mecarbinzid); carbanilate fungicides (e.g., diethofencarb); conazole fungicides (imidazoles) climbazole, clotrimazole, imazalil, oxpoconazole, prochloraz, triflumizole); conazole fungicides (e.g., azaconazole, brumoconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P); copper fungicides (e.g., Bordeaux mixture, Burgundy mixture, Cheshunt mixture; copper acetate, basic copper, copper hydroxide, copper naphthenate, copper oleate, copper oxychloride, copper sulfate, basic copper sulfate, copper zinc chromate, cufraneb, cuprobam, cuprous oxide, mancopper, oxine copper); dicarboximide fungicides (e.g., famoxadone, fluoroimide); dichlorophenyl dicarboximide fungicides (e.g., chlozolinate, dichlozoline, iprodione, isovaledione, myclozolin, procymidone, and vinclozolin); phthalimide fungicides (e.g., captafol, captan, ditalimfos, folpet, thiochlorfenphim); dinitrophenol fungicides (e.g., binapacryl, dinobuton, dinocap, dinocap-4, dinocap-6, dinocton, dinopenton, dinosulfon, dinoterbon, DNOC), dithiocarbamate fungicides (e.g., azithiram, carbamorph, cufraneb, cuprobam, disulfiram, ferbam, metam, nabam, tecoram, thiram, ziram); cyclic dithiocarbamate fungicides (e.g., dazomet, etem, milneb); polymeric dithiocarbamate fungicides (e.g., mancopper, mancozeb, maneb, metiram, polycarbamate, propineb, zineb); imidazole fungicides (e.g., cyazofamid, fenamidone, fenapanil, glyodin, iprodione, isovaledione, pefurazoate, triazoxide); inorganic fungicides (e.g., potassium azide, potassium thiocyanate, sodium azide, sulfur); inorganic mercury fungicides (e.g., mercuric chloride, mercuric oxide, mercurous chloride); organomercury fungicides (e.g., (3-ethoxypropyl) mercury bromide, ethylmercury acetate, ethylmercury bromide, ethylmercury chloride, ethylmercury 2,3-dihydroxypropyl mercaptide; ethylmercury phosphate; N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivate of pyrocatechol, phenylmercury nitrate, phenylmercury salicylate, thiomersal, tolylmercury acetate); morpholine fungicides (e.g., aldimorph, benzamorf, carbamorph, dimethomorph, dodemorph, fenpropimorph, flumorph, tridemorph); organophosphorus fungicides (e.g., ampropylfos, ditalimfos, edifenphos, fosetyl, hexylthiofos, iprobenfos, phosdiphen, pyrazophos, tolclofos-methyl, tramiphos), organotin fungicides (e.g., decafentin, fentin, tributyltin oxide); oxathiin fungicides (e.g., carboxin, oxycarboxin); oxazole fungicides (e.g., clhozolinate, dichlozoline, drazoxolon, famoxadone, hymexazol, metazoxolon, myclozolin, oxadixyl, vinclozolin); polysulfide fungicides (e.g., barium polysulfide, calcium polysulfide, potassium polysulfide, sodium polysulfide); pyrazole fungicides (e.g., furametpyr, penthiopyrad); pyridine fungicides (e.g, boscalid, buthiobate, dipyrithione, fluazinam, fluopicolide, pyridinitril, pyrifenox, pyroxychlor, pyroxyfur); pyrimidine fungicides (e.g., bupirimate, cyprodinil, diflumetorim, dimethirimol, ethirimol, fenarimol, ferimzone, mepanipyrim, nuarimol, pyrimethanil, triarimol); pyrrole. fungicides (e.g., fenpiclonil, fludioxonil, fluoroimide); quinoline fungicides (e.g., ethoxyquin, halacrinate, 8-hydroxyquinoline sulfate), quinacetol, quinoxyfen); quinone fungicides (e.g., benquinox, chloranil, dichlone, dithianon); quinoxaline fungicides (e.g. chinomethionat, chlorquinox, thioquinox); thiazole fungicides (e.g., ethaboxam, etridiazole, metsulfovax, octhilinone, thiabendazole, thiadifluor, thifluzamide); thiocarbamate fungicides (e.g., methasulfocarb, prothiocarb); thiophene fungicides (e.g., ethaboxam, silthiofam); triazine fungicides (e.g., anilazine); triazole fungicides (bitertanol, fluotrimazole, triazbutil); urea fungicides (e.g., bentaluron, pencycuron, quinazamid); unclassified fungicides (e.g., acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, probenazole, proquinazid, pyroquilon, sodium orthophenylphenoxide, spiroxamine, sultropen, thicyofen, tricyclazole, zinc naphthenate).

Examples of BASs that are bacteriocides include: bronopol; copper hydroxide; cresol; dichlorophen; dipyrithione; dodicin; fenaminosulf; formaldehyde; hydrargaphen; 8-hydroxyquinoline sulfate; kasugamycin; nitrapyrin; octhilinone; oxolinic acid; oxytetracycline; probenazole; streptomycin; tecloftalam; and thiomersal.

Examples of general classes of BASs that are pesticides include: acaricides; algaecides; antifeedants; avicides; bacteriocides; bird repellents; chemosterilants; fungicides; herbicide safeners; herbicides; insect attractants; insect repellents; insecticides; mammal repellents; mating disruptors; molluscicides; nematicides; plant activators; plant growth regulators; rodenticides; synergists; and, virucides.

Examples of BASs that are algaecides include: bethoxazin; copper sulfate; cybutryne; dichlone; dichlorophen; endothal; fentin; hydrated lime; nabam; quinoclamine; quinonamid; and, simazine.

Examples of BASs that are molluscicides include: bromoacetamide; calcium arsenate; cloethocarb; copper acetoarsenite; copper sulfate; fentin; metaldehyde; methiocarb; niclosamide; pentachlorophenol; sodium pentachlorophenoxide; tazimcarb; thiodicarb; tributyltin oxide; trifenmorph; and, trimethacarb.

Ceramic structures of the present invention typically include solid, porous oxides of titanium, zirconium, tantalum, scandium, cerium, and yttrium, either individually or as mixtures. Preferably, the ceramic is a titanium oxide or a zirconium oxide, with titanium oxides being especially preferred. Structural characteristics of the ceramics are well-controlled, either by synthetic methods or separation techniques. Examples of controllable characteristics include: 1) whether the structure is roughly spherical and hollow, non-spherical and hollow, or a collection of smaller particles bound together in approximately spherical shapes or non-spherical shapes; 2) the range of structure sizes (e.g., particle diameters); 3) surface area of the structures; 4) wall thickness, where the structure is hollow; and, 5) pore size range.

The ceramics are typically produced by spray hydrolyzing a solution of a metal salt to form particles, which are collected and heat treated. Spray hydrolysis initially affords noncrystalline spheres. The surface of the spheres consists of an amorphous, glass-like film of metal oxide or mixed-metal oxides. Calcination, or heat treatment, of the material causes the film to crystallize, forming an interlocked framework of crystallites. The calcination products are typically porous, rigid structures. (See, for example, U.S. Pat. No. 6,375,923, which is incorporated-by-reference for all purposes.)

A variety of roughly spherical ceramic materials are produced through the variation of certain parameters: a) varying the metal composition or mix of the original solution; b) varying the solution concentration; and, c) varying calcinations conditions. Furthermore, the materials can be classified according to size using well-known air classification and sieving techniques.

In the case of roughly spherical, hollow structures, particle sizes typically range from 1 μm to 100 μm in diameter. The particle diameter often times ranges from 3μ to 50 μm, with 5 μm to 25 μm being preferred.

Surface area of the ceramic structures depends on several factors, including particle shape, particle size, and particle porosity. Typically, the surface area of roughly spherical particles ranges from 0.1 m²/g to 100 m²/g. The surface area oftentimes, however, ranges from 0.5 m²/g to 50 m²/g.

Wall thicknesses of hollow particles tend to range from 10 nm to 5 μm, with a range of 50 nm to 3 μm being typical. Pore sizes of such particles further range from 1 nm to 5 μm, and oftentimes lie in the 5 nm to 3 μm range.

Without further treatment, the ceramic structures of the present invention are hydrophilic. The degree of hydrophilicity, however, may be chemically modified using known techniques. Such techniques include, without limitation, treating the structures with salts or hydroxides containing magnesium, aluminum, silicon, silver, zinc, phosphorus, manganese, barium, lanthanum, calcium, cerium, and PEG polyether or crown ether structures. Such treatments influence the ability of the structures to uptake and incorporate BASs, particularly hydrophilic drugs, within their hollow space.

Alternatively, the structures may be made relatively hydrophobic through treatment with suitable types of chemical agents. Hydrophobic agents include, without limitation, organo-silanes, chloro-organo-silanes, organo-alkoxy-silanes, organic polymers, and alkylating agents. These treatments make the structures more suitable for the incorporation of lipophilic or hydrophobic BASs (e.g., drugs). Additionally, the porous, hollow structures may be treated using chemical vapor deposition, metal vapor deposition, metal oxide vapor deposition, or carbon vapor deposition to modify their surface properties.

The BAS (e.g., drug) that is applied to the ceramic structures may optionally include an excipient. Examples of excipients include, without limitation, the following: acetyltriethyl citrate; acetyl tri-n-butyl citrate; aspartame; aspartame and lactose; alginates; calcium carbonate; carbopol; carrageenan; cellulose; cellulose and lactose combinations; croscarmellose sodium; crospovidone; dextrose; dibutyl sebacate; fructose; gellan gum, glyceryl behenate; magnesium stearate; maltodextrin; maltose; mannatol; carboxymethylcellulose; polyvinyl acetate phathalate; povidone; sodium starch glycolate; sorbitol; starch; sucrose; triacetin; triethylcitrate; and, xanthan gum.

A BAS (e.g., drug) may be combined with a ceramic structure of the present invention using any suitable method, although solvent application/evaporation and drug melt are preferred. For solvent application/evaporation, a BAS (e.g., drug) of choice is dissolved in an appropriate solvent. Such solvents include, without limitation, the following: water, buffered water, an alcohol, esters, ethers, chlorinated solvents, oxygenated solvents, organo-amines, amino acids, liquid sugars, mixtures of sugars, supercritical liquid fluids or gases (e.g., carbon dioxide); hydrocarbons, polyoxygenated solvents, naturally occurring or derived fluids and solvents, aromatic solvents, polyaromatic solvents, liquid ion exchange resins, and other organic solvents. The dissolved BAS (e.g., drug) is mixed with the porous ceramic structures, and the resulting suspension is degassed using pressure swing techniques or ultrasonics. While stirring the suspension, solvent evaporation is conducted using an appropriate method (e.g., vacuum, spray drying under low partial pressure or atmospheric pressure, and freeze drying).

Alternatively, the above-described suspension is filtered, and the coated ceramic particles are optionally washed with a solvent. The collected particles are dried according to standard methods. Another alternative involves filtering the suspension and drying the wet cake using techniques such as vacuum drying, air stream drying, microwave drying and freeze-drying.

For the BAS (e.g., drug) melt coating method, a melt of the desired BAS is mixed with the porous, hollow ceramic structures under low partial pressure conditions (i.e., degassing conditions). The mix is allowed to equilibrate to atmospheric pressure and to cool under agitation. This process affords a powder with drug both inside and outside the structures. BAS (e.g., drug) may be removed from the particle surface prior to tableting by simple washing of the particle surface with an appropriate solvent and subsequent drying.

BAS (e.g., drug) on the inside or outside of the ceramic structures is typically coated in a thickness ranging from 10 nm to 10 μm, with 50 nm to 5 μm being preferred. The corresponding weight ratio of drug to particle usually ranges from 1.0 to 100, with a range of 2.0 to 50 being preferred.

Coated BAS (e.g., drug) may exist in either a crystalline or amorphous (noncrystalline) form. Crystalline materials exhibit characteristic shapes and cleavage planes due to the arrangement of their atoms, ions or molecules, which form a definite pattern called a lattice. An amorphous material does not have a molecular lattice structure. This distinction is observed in powder diffraction studies of materials: In powder diffraction studies of crystalline materials, peak broadening begins at a grain size of about 500 nm. This broadening, continues as the crystalline material gets small until the peak disappears at about 5 nm. By definition, a material is “amorphous” by XRD when the peaks broaden to the point that they are not distinguishable from background noise, which occurs at 5 nm or smaller.

The coated BAS (e.g., drug) on the particle is in a substantially pure form. Typically, the BAS is at least 95.0% pure, with a purity value of at least 97.5% being preferred and a value of at least 99.5% being especially preferred. In other words, BAS (e.g., drug) degradants (e.g., hydrolysis products, oxidation products, photochemical degradation products, etc.) are kept below 5.0%, 2.5%, or 0.5% respectively.

The BAS containing materials typically include a semi-impermeable membrane (e.g., porous hydrophobic or hydrophilic polymer) that imparts controlled release characteristics to the materials. The semi-impermeable membrane may either be applied after the BAS is combined, in which it serves as a coating overtop the BAS, or it may be applied before the BAS is combined. In either case, the release (e.g., delivery) rate is decreased due to the increased time needed for BAS (e.g., drug) molecules to diffuse through the membrane.

The semi-permeable membrane may either be coated on the outside of the material, as noted above, or impregnated within it. Where it is impregnated, the method of application is typically through pressure optimized polymer embedding (i.e., POPE™). This method involves contacting the material with a polymer in liquid or semisolid form, and varying pressure to force the polymer into the pores of the materials. In certain cases, negative pressure is employed; in others positive pressure is used.

Examples of hydrophobic polymers that may be applied to the combination of the present invention include, without limitation, the following: an alkylcellulose polymer (e.g., ethylcellulose polymer); and, an acrylic polymer (e.g., acrylic acid and methacrylic acid copolymers, methacrylic acid copolymers, methyl methacrylate copolymers, ethoxyethyl methacrylates, cyanaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers).

Where the BAS is a drug, the drug containing materials may optionally include a second or third drug or prodrug. A nonlimiting example of such a second drug is a cytochrome P450 inhibitor (e.g., ketoconazole and isoniazid). The materials may further be optionally coated with a variety of sugars or even polymers, typically hydrophilic or hydrophobic organic polymers, other than those of semi-permeable membranes.

A drug/ceramic structure combination of the present invention, which includes a semi-impermeable membrane or possesses an appropriate pore size, provides for sustained delivery of the drug to the patient when administered to a patient. Typically, when the subject combination is tested using the USP Paddle Method at 100 rpm in 900 ml aqueous buffer (pH between 1.6 and 7.2) at 37° C., the following dissolution profile will be provided: between 5.0% and 50.0% of the drug released after 1 hour; between 10.0% and 75.0% of the drug released after 2 hours; between 20.0% and 85.0% of the drug released after 4 hours; and, between 25.0% and 95.0% of the drug released after 6 hours. Oftentimes, from hour 1 until hour 4, 5 or 6, drug release is observed to follow zero-order kinetics.

Where the drug/ceramic structure combination of the present invention does not contain the optional polymer coating or pores of an appropriate size, the rate of drug delivery is actually increased over a solid form of the drug itself. It is hypothesized that this rate increase is primarily due to the increased surface area of the drug, which, in turn, increases its dissolution rate. Typically, when the combination—absent the second coating—is tested using the USP Paddle Method discussed above, the ratio of drug dissolution rate from the combination to the dissolution rate for the same amount of drug in tablet form is at least 1.1. Preferably, the ratio is at least 1.5. More preferably it is at least 2.0 and most preferably at least 3.0. This combination is especially useful for the delivery of drugs with solubilities less than 1.0 mg/ml of water.

When the drug/ceramic structure combination is administered to a patient in need of treatment, the drug dosage is typically in the range from 100 ng to 1 g, preferably 1 mg to 750 mg. The exact dosage will depend on the particular drug in the combination, and can be determined using well-known methods.

The drug/ceramic structure combinations exhibit beneficial stability characteristics under a number of conditions. In other words, the included drug does not substantially decompose over reasonable periods of time. At 25° C. over a two week period for instance, the drug purity typically degrades less than 5%. Oftentimes, there is less than 4%, 3%, 2%, or 1% degradation (e.g., hydrolysis, oxidation, photochemical reactions).

Where the BAS is not a drug, the BAS/ceramic structure combination is typically included in a coating, such as paint. Such coatings can provide a barrier that is resistant to fungus, mildew, bacteria, etc. Thereby, the coated object is protected.

The following examples are meant to illustrate the present invention and are not meant to limit it in any way.

### EXAMPLE 1

An aqueous solution of titanium oxychloride and HCl containing 15 g/l Ti and 55 g/l Cl was injected in a titanium spray drier at a rate of 12 liters/h. The outlet temperature from the spray drier was 250° C. A solid intermediate product consisting of amorphous spheres was recovered on a bag filter. The intermediate product was calcined in a muffle furnace at 950° C. for 8 h. The calcined material was further classified by passing it through a set of cyclones. The size fraction 15-25 μm was screened. X-Ray diffraction shows that product is made primarily of TiO₂ rutile, with about 1% anatase. The average mechanical strength of the particles was measured by placing a counted number of them on a flat metal surface, positioning another metal plate on top and progressively applying pressure until the particles begin to break. Scanning electron micrographs of the calcined product show that it is made of rutile crystals, bound together as a thin-film structure. The thickness of the film is about 500 nm and the pores have a size of about 50 nm.

### EXAMPLE II

The experiment of example I was repeated at different calcining temperatures over the range 500 to 900° C., with different concentrations of chloride and titanium in solution. The titanium concentration was varied over the range 120 to 150 g/l Ti. In general, a higher temperature creates larger and stronger particles, a lower Ti concentration tends to decrease the size of the spheres, to increase the thickness of the walls and to increase the mechanical strength of the particles.

### EXAMPLE III

The conditions were the same as those of Example I, except that a eutectic mixture of chloride salts of Li, Na and K equivalent to 25% of the amount of TiO₂ present was added to the solution before the spraying step and a washing step was added after the calcination step. In the washing step, the calcined product was washed in water and the alkali salts were thereby removed from the final product. The advantage of using the salt addition is that the spheres of the final product have a thicker wall. Additionally, the non-reactive or nearly non-reactive salt produces salt grains in the wall of the ceramic structure after calcinations at below reactive temperatures. These salt grains are easily dissolved by immersion in water. After washing and drying, voids appear in the wall of the ceramic structure. These voids are pores through which the drug may be accessed. Using different salts or salt mixtures results in different sized salt grains after calcinations, and therefore offers pore size control. Salts include alkaline and alkaline earth metal chlorides.

### EXAMPLE IV

The conditions were the same as those of Example I, except that an amount of sodium phosphate Na₃PO₄ equivalent to 3% of the amount of TiO₂ present was added to the solution before spraying. The additive ensured faster rutilization of the product during calcination. The final product produced in this example consisted of larger rutile crystals than in the other examples, and exhibited a higher mechanical strength.

### EXAMPLE V

Example IV was repeated in different conditions of temperature and concentration and with different compounds serving as ligands. The following compounds were used as ligands: proteins, enzymes; polymers; colloidal metals, metal oxides and salts; active pharmaceutical ingredients. Temperatures are adapted to take into account the stability of the ligands. With organic compounds, the temperature is generally limited to about 150° C.

### EXAMPLE VI

Titanium oxychloride solution is prepared from TiCl₄, HCl and water by controlled addition rate of TiCl₄ into a well-mixed and temperature-controlled concentrated HCl solution. To the clear solution is added a surface tension reducing agent, which produces smaller droplets and therefore smaller ceramic structures during spraying in this environment. These detergents include alkali phosphates/pyrophosphates and acid phosphates. Also, a particle size or shape control agent is dissolved in the clear solution. Both functions (surface tension reduction and Rutilizing agent) are supplied by Na₃PO₄. The Na₃PO₄ is added at 3 wt %, TiO₂ basis. The solution is spray dried in a Titanium lined spray dryer with a rotary atomizer at a 250° C. discharge temperature. The collected powder is amorphous by XRD, generally spherical in shape, and, for the most part, hollow. The collected powder is 4 wt % volatiles at 800° C. The volatiles are 20% HCl and 80% water. The amorphous powder is calcined at 700° C., in a tray in an oven for 6 hours. A ceramic structure is produced with a lattice work of TiO₂ crystals. The ceramic structure is then soaked in an HCl solution, washed and dried in an oven. This removes the non-reactive control agents. The ceramic structure is then annealed in a try in an oven by heating to 800° C. and soaked at that temperature for 6 hours. The crystal substructure is thereby “glassed,” fused, and strengthened. The annealed ceramic structures are then sized by screening to ˜20 μm producing a population primarily between 5 μm and 20 μm. The sized and annealed ceramic structures are then treated with a hydrophobizing agent (as previously mentioned) and thermally treated. A hydrophobic ceramic surface is produced. A solution of drug and alcohol are added to the ceramic structures and pressured to assure good fill. Excess solution is drained off. The mixture of ceramic structures and drug solution is then vacuum dried.

### EXAMPLE VII

Dry Classification: R172Cd (½) USP-DC of titanium particles (25.14 m²/gm) and R226DC3 ( 2/2) VHP-DC of titanium particles (24.82 m²/gm), were provided. Material was classified using stainless steel Tyler sieves (Nos. 325 and 400) agitated for approximately 15 min using a shaker table. (No solvents were used to aid in the classification of the material.) Experimental results were assessed visually using SEM images (see Table 1).

TABLE 1
Dry Screening USP-VHP Results

| | | VHP | VHP Mass | | USP Mass |
|---|---|---|---|---|---|
| Tyler Mesh | Particle Size, | Mass | Recovered | USP Mass | Recovered |
| No. | d, (μm) | (gm) | (%) | (gm) | (%) |
| 325 | d ≧ 45 | 24.93 | 60.4 | 9.58 | 39.4 |
| 400 | 45 > d ≧ 38 | 7.84 | 19.0 | 2.99 | 12.3 |
| — | 38 > d | 8.52 | 20.6 | 11.74 | 48.3 |
| Total | — | 41.29 | 100.0 | 24.31 | 100.0 |

### EXAMPLE VIII

Wet Classification: A small sample of USP material was screened using Tyler screen Nos. 325 400, 450, 500 and 635. Results were determined using SEM imaging. Table 2 tabulates results from the experiment (see Table 2).

TABLE 2
Particle Size Data for 6 gm USP Sample

| | | Particle Size, | | Cumulative |
|---|---|---|---|---|
| Sample No. | Tyler Mesh No. | d, (μm) | Mass (gm) | Percent (%) |
| 1 | 325 | d ≧ 45 | 2.27 | 36.9 |
| 2 | 400 | 45 > d ≧ 38 | 0.33 | 42.2 |
| 3 | 450 | 38 > d ≧ 32 | 0.36 | 48.1 |
| 4 | 500 | 32 > d ≧ 25 | 0.40 | 54.5 |
| 5 | 635 | 25 > d ≧ 20 | 0.57 | 63.8 |
| 6 | — | 20 > d | 2.23 | 100 |
| Total | — | — | 6.16 | — |

### EXAMPLE IX

Biologically Active Substance Loading: A solution of quinine in reagent alcohol (0.37 gm/mL) was dripped onto unscreened USP titanium particles. The USP material was allowed to “wick” or take up the liquid without producing visible free liquid in the mixture. After the solid had become saturated with solution, the material was dried in a drying oven at approximately 110° C. Using a balance, the weight of the loaded titanium particle was taken until the weight stabilized. After the weight had stabilized, the final weight was compared to the initial weight to determine the percent loading of the material. This was repeated for separate samples for 1, 2, and 3 loadings. Afterward, the samples were collected and analyzed for specific gravity and tap density (see Table 4 for results).

TABLE 4
Biologically Active Substance Loading by Wicking the Material

| | | | | Change in | | |
|---|---|---|---|---|---|---|
| | | | Initial Mass | Titanium | | |
| | | Total Volume | of Titanium | Nanosphere | Specific | Tap |
| Sample | No. of | of Solution | Nanosphere | Mass After | Gravity | Density |
| No. | Loadings | Loaded (mL) | (gm) | Loading (gm) | (gm/cm³) | (gm/cm³) |
| 0 | 0 | 6.4* | 3.007 | No change | 4.34 | 0.43 |
| 1 | 1 | 5.2 | 3.002 | 1.348 | 2.37 | 0.59 |
| 2 | 2 | 8.3 | 2.998 | 2.432 | 1.98 | 0.54 |
| 3 | 3 | 12.4 | 3.004 | 3.608 | 1.87 | 0.56 |

*Denotes a blank sample. The solution loaded was reagent alcohol with no BAS.

### EXAMPLE X

USP grade BAS was loaded under vacuum using a Rotovapor R-200 (Rotovap) retrofitted with PTFE tubing, a glass capillary, and Teflon agitators. 10 g of 38 μm titanium particle USP material was placed in an evaporation flask. The loaded evaporation flask is attached to the Rotovap, and the Rotovap was turned on. All Rotovap openings were closed and th evaporation flask was rotated. The titanium nanosphere material was evacuated for approximately 1 h.

A 15 mL solution of 0.35 gm/mL quinine in 200 proof ethanol solution was measured and placed in a 50 mL graduated cylinder. The pump was turned off, and the sample was exposed to the atmosphere. After the flask pressure equalized to atmosphere, the pump was turned on, and a slight vacuum was created in the Rotovap. The external PTFE tubing was placed in the graduated cylinder, and the Rotovap vacuum was used to pull the solution into the flask. The tubing was cleaned using 2 mL of ethanol using the same method as injection. The solution and titanium nanospheres were allowed to mix for 1 h. After 30 min. of mixing, a slight vacuum was applied intermittently to the flask. The Rotovap flask was rotated, and vacuum was applied to dry material. Heat was applied using the Rotovap bath to remove any entrained ethanol. The final weight of the flask and sample was submitted for SEM and carbon analysis. Table 5 provides experimental carbon analysis results.

TABLE 5
Carbon and Quinine Loading Data (1 Loading)

| | Percent | Mass | Moles of | Moles of | Grams of | Percent |
|---|---|---|---|---|---|---|
| Sample | Carbon | Carbon | Carbon | Quinine | Quinine | Quinine |
| No. | (%) | (gm) | (mol) | (mol) | (gm) | Loaded (%) |
| 1 | 18.59 | 0.1859 | 1.548 × 10⁻² | 7.739 × 10⁻⁴ | 0.2511 | 25.11 |
| 2 | 17.28 | 0.1728 | 1.439 × 10⁻² | 7.194 × 10⁻⁴ | 0.2334 | 23.34 |
| 3 | 18.65 | 0.1865 | 1.553 × 10⁻² | 7.764 × 10⁻⁴ | 0.2519 | 25.19 |
| 5 | 0.415 | 4.15 × 10⁻³ | 3.46 × 10⁻⁴ | 1.73 × 10⁻⁵ | 5.61 × 10⁻³ | 0.56 |

1. A composition for sustained delivery of a biologically active substance, wherein the composition comprises a biologically active substance and a ceramic structure, and wherein the ceramic structure comprises a metal oxide selected from a group consisting of titanium oxide, zirconium oxide, scandium oxide, cerium oxide and yttrium oxide.

2. The composition according to claim 1, wherein, the biologically active substance is selected from a group consisting of: a drug, a biocide, a mildewcide, a fungicide, a bacteriocide, an algaecides, and mixtures thereof.

3. The composition according to claim 2, wherein the biologically active substance is a drug selected from a group consisting of: antipyretics, analgesics, antiphlogistics, steroidal anti-inflammatories, coronary vasodilators, peripheral vasodilators, antibiotics, synthetic antimicrobials, antiviral agents, anticonvulsants, antitussives, expectorants, bronchodilators, cardiacs, muscle relaxants, cerebral metabolism ameliorants, minor tranquilizers, major tranquilizers, betablockers, antiarrythmics, anthrifuges, anticoagulants, thrombolytics, antihistamines, antiemetics, depressors, hyperlipidemia agents, sympathetic nervous stimulants, oral diabetes therapeutics, oral carcinostatics, vitamins, thamuria therapeutics, and angiotensin convertase inhibitors.

4. The composition according to claim 1, wherein the ceramic structure comprises either a hollow portion wherein the biologically active substance is included in the hollow portion or a collection of smaller particles bound together.

5. The composition according to claim 1, wherein the ceramic structure is roughly spherical.

6. The composition according to claim 5, wherein the roughly spherical structure has a diameter, and wherein the diameter ranges from 1 μm to 100 gm.

7. The composition according to claim 6, wherein the diameter ranges from 3 μm to 50 μm.

8. The composition according to claim 1, wherein the ceramic structure comprises pores, and wherein the pores have diameters, and wherein the pore diameters range from 1 nm to 5 μm.

9. The composition according to claim 1, wherein there ceramic structure has been treated with a salt or a hydroxide.

10. The composition according to claim 9, wherein the salt or hydroxide comprises magnesium, aluminum, silicon, silver, zinc, phosphorus, manganese, barium, lanthanum, calcium cerium, PEG polyethers, or crown ethers.

11. The composition according to claim 1, wherein the ceramic structure has been treated with a hydrophobic agent.

12. The composition according to claim 11, wherein the hydrophobic agent is selected from a group consisting of an organo-silane, a chloro-organo-silane, an organoalkoxy silane, an organic polymer, and an alkylating agent.

13. The composition according to claim 1, wherein the combination further comprises an excipient.

14. The composition according to claim 1, wherein the biologically active substance coats the ceramic structure in a thickness range, and wherein the thickness ranges from 10 nm to 10 μm.

15. The composition according to claim 14, wherein the coated biologically active substance is in an amorphous form.

16. The composition according to claim 14, wherein the coated biologically active substance is in a crystalline form.

17. A process for preparing a composition according to claim 1, wherein the process comprises:
a) dissolving the biologically active substance in a solvent to provide a solution;
b) contacting the solution with the ceramic structure; and,
c) evaporating the solvent
thereby providing the composition.

18. The process according to claim 17, wherein the process further comprises: degassing a suspension that results from contacting the solution with the ceramic structure.

19. The process according to claim 17, wherein the process further comprises removing any biologically active substance coated on the outside of the ceramic structure.

20. The process according to claim 17, wherein the solvent is selected from a group consisting the following: water, buffered water, an alcohol, esters, ethers, chlorinated solvents, oxygenated solvents, organo-amines, amino acids, liquid sugars, mixtures of sugars, supercritical liquid fluids or gases, hydrocarbons, polyoxygenated solvents, naturally occurring or derived fluids and solvents, aromatic solvents, polyaromatic solvents, liquid ion exchange resins, and other organic solvents.
